(19) United States
(12) Patent Application Publication
(10) Pub. No.: US 2026/0026858 A1
(43) Pub. Date: Jan. 29, 2026

(54) STEERABLE ASSEMBLY FOR TREATING A VERTEBRAL BODY

(71) Applicant: Relievant Medsystems, Inc., Redwood City, CA (US)

(72) Inventors: Benjamin David Parker, Sunnyvale, CA (US); Neil Shigeo Sasaki, Edina, MN (US); Michael Makay, Stafford, VA (US)

(21) Appl. No.: 19/277,207

(22) Filed: Jul. 22, 2025

Related U.S. Application Data
(60) Provisional application No. 63/676,170, filed on Jul. 26, 2024.

(51) Int. Cl. A61B 18/14; A61B 18/00

(52) U.S. Cl. CPC A61B 18/14; A61B 2018/00077; A61B 2018/00184; A61B 2018/00339; A61B 2018/00434; A61B 2018/0091

(57) ABSTRACT

A system for treating a basivertebral nerve in a vertebral body of a patient includes an introducer having a central channel and a steerable assembly sized for passable through the central channel. The introducer is configured to pass through the cortical bone of the vertebral body and into the cancellous bone of the vertebral body, and the steerable assembly has a first configuration and a deflected configuration. The steerable assembly includes an elongate member and a hypotube over a portion of the elongate member. The elongate member has a proximal end and a distal portion and is deflectable between the first configuration and the deflected configuration, and the hypotube has a proximal end and a distal end. The distal portion of the elongate member is configured to engage the distal end of the hypotube.

### CROSS REFERENCE TO RELATED APPLICATIONS

The present application claims priority to U.S. Provisional Patent Application No. 63/676,170, filed Jul. 26, 2024, the disclosure of which is incorporated herein in its entirety.

### TECHNICAL FIELD

Described herein are various implementations of systems and methods for modulating tissue. More specifically, the disclosure relates to systems for accessing and treating locations within vertebral bodies, for example, by ablating nerves or other tissue within or surrounding a vertebral body to treat chronic lower back pain.

### BACKGROUND

Back pain is a very common health problem worldwide and is a major cause for work-related disability benefits and compensation. At any given time, low backpain impacts nearly 30% of the US population, leading to 62 million annual visits to hospitals, emergency departments, outpatient clinics, and physician offices. Back pain may arise from strained muscles, ligaments, or tendons in the back and/or structural problems with bones or spinal discs, including vertebral endplate degeneration or defects (e.g., pre-Modic changes). The back pain may be acute or chronic. Existing treatments for chronic back pain vary widely and include physical therapy and exercise, chiropractic treatments, injections, rest, pharmacological therapy such as opioids, pain relievers or anti-inflammatory medications, and surgical intervention such as vertebral fusion, discectomy (e.g., total disc replacement), or disc repair. Existing treatments can be costly, addictive, temporary, ineffective, and/or can increase the pain or require long recovery times. In addition, existing treatments do not provide adequate relief for the majority of patients and only a small percentage are surgically eligible.

### SUMMARY

In Example 1, a system for treating a basivertebral nerve in a vertebral body of a patient, the system comprising a steerable assembly sized for passage through the central channel of an introducer, the steerable assembly having a first configuration and a deflected configuration, the steerable assembly including an elongate member having a proximal end and a distal portion, wherein the distal portion of the elongate member is deflectable between the first configuration and the deflected configuration, and a hypotube disposed over a portion of the elongate member, the hypotube comprising a proximal end and a distal end.

In Example 2, the system of Example 1, wherein when the distal portion of the elongate member is in engagement with the distal end of the hypotube, axial movement of one of the elongate member and the hypotube relative to the other of the elongate member and the hypotube causes the distal ends of the elongate member and the hypotube to move from their respective first configurations to their respective deflected configurations.

In Example 3, the system of any of Examples 1 and/or 2, wherein the distal portion of the elongate member has a pre-formed curve in the first configuration.

In Example 4, the system of Examples 1 and/or 2 and 3, wherein the pre-formed curve is less curved than the deflected configuration of the elongate member.

In Example 5, the system of any of Examples 1-4, wherein at least the distal end of the elongate member is deflectable between a straight configuration and the deflected configuration.

In Example 6, the system of any of Examples 1-5, wherein at least the distal end of the hypotube is deflectable between a straight configuration and the curved configuration.

In Example 7, the system of any of Examples 1-6, wherein a distal deflectable section of the hypotube includes a plurality of slits.

In Example 8, the system of any of Examples 1-6 and claim 7, wherein the plurality of slits is on one side of the hypotube opposite of a solid heel.

In Example 9, the system of any of Examples 1-6 and claim 7 wherein each of the plurality of slits extends through a majority of the circumference of the hypotube and has ends that are oriented radially with respect to a central axis of the hypotube.

In Example 10, the system of any of Examples 1-9, wherein the hypotube is formed at least in part from stainless steel.

In Example 11, the system of Examples 1-10, wherein the elongate member defines a plurality of notches at the elongate member distal portion to facilitate deflection of the distal portion of the elongate member between the first configuration and the deflected configuration, and the distal portion of the elongate member defines a longitudinal axis and first and second cylindrical sections defined by a plane extending along the longitudinal axis, and wherein the plurality of notches are defined in the first cylindrical section.

In Example 12, the system of any of Examples 1-11, wherein the distal portion of the elongate member defines a shoulder and wherein the distal end of the hypotube engages the shoulder of the elongate member.

In Example 13, the system of any of Examples 1-12, further comprising a first electrode and a second electrode configured to deliver bipolar radiofrequency energy to a treatment location.

In Example 14, the system of any of Examples 1-12 and Example 13, further comprising a cannulated probe sized for passage through the central channel of the introducer, the cannulated probe configured to be slidably received over the elongate member, the cannulated probe having a proximal end and a distal end wherein the first electrode and the second electrode are located proximate the cannulated probe distal end.

In Example 15, the system of any of Examples 1-12 and Example 13, wherein the elongate member comprises the first electrode and wherein the hypotube comprises the second electrode.

In Example 16, a system for treating a basivertebral nerve in a vertebral body of a patient, the system comprising an introducer having a central channel, the introducer configured to pass through the cortical bone of the vertebral body and into the cancellous bone of the vertebral body, a steerable assembly sized for passage through the central channel of the introducer, the steerable assembly including an elongate member having a proximal end and a distal portion, wherein the distal portion of the elongate member is deflectable between the first configuration and the deflected configuration, and a hypotube disposed over a portion of the elongate member, the hypotube comprising a proximal end and a distal end, wherein the distal portion of the elongate member is configured to engage the distal end of the hypotube, wherein axial translation of one of the elongate member and the hypotube relative to the other of the elongate member and the hypotube causes the steerable assembly to move from the first configuration to the deflected configuration.

In Example 17, the system of Example 16, wherein the hypotube is formed at least in part from stainless steel.

In Example 18, the system of Example 16, wherein the elongate member defines a plurality of notches at the elongate member distal portion to facilitate deflection of the distal portion of the elongate member between the first configuration and the deflected configuration, and the distal portion of the elongate member defines a longitudinal axis and first and second cylindrical sections defined by a plane extending along the longitudinal axis, and wherein the plurality of notches are defined in the first cylindrical section.

In Example 19, the system of Example 16, wherein the distal portion of the elongate member defines a shoulder and wherein the distal end of the hypotube engages the shoulder of the elongate member.

In Example 20, the system of Example 16, wherein the distal end of the elongate member has a pre-formed curve in the first configuration.

In Example 21, the system of Example 16, further comprising a first electrode and a second electrode configured to deliver bipolar radiofrequency energy to a treatment location.

In Example 22, the system of Example 21, further comprising a cannulated probe sized for passage through the central channel of the introducer, the cannulated probe configured to be slidably received over the elongate member, the cannulated probe having a proximal end and a distal end, wherein the first electrode and the second electrode are located proximate the cannulated probe distal end.

In Example 23, the system of Example 21, wherein the elongate member comprises the first electrode and wherein the hypotube comprises the second electrode.

In Example 24, a method of treating a basivertebral nerve in a vertebral body of a patient, the method comprising inserting an introducer through the cortical bone of the vertebral body and into a cancellous bone region of the vertebral body, the introducer having a shaft defining a central channel and an axial opening in communication with the central channel located at a distal end of the shaft, advancing a steerable assembly through the central channel of the introducer, the steerable assembly comprising an elongate member having a proximal end and a deflectable distal portion, and a hypotube that extends over the elongate member such that a distal end of the hypotube is in engagement with the distal portion of the elongate member, extending the steerable assembly beyond the axial opening of the introducer shaft, and axially moving one of the elongate member and the hypotube relative to the other of the elongate member and the hypotube to cause the distal deflectable sections of the elongate member and the hypotube to deflect away from a central axis of the introducer shaft.

In Example 25, the method of Example 24, wherein the operation of axially moving one of the elongate member and the hypotube relative to the other of the elongate member and the hypotube causes the distal deflectable section of the elongate member to channel within the cancellous bone region.

In Example 26, the method of Example 24, wherein the operation of axially moving one of the elongate member and the hypotube relative to the other of the elongate member and the hypotube comprises pulling the elongate member in a proximal direction relative to the hypotube.

In Example 27, the method of Example 24, further comprising retracting the hypotube through the central channel of the introducer over the elongate member, and advancing a cannulated probe through the central channel of the introducer, over the elongate member, and along a path in the cancellous bone region until a distal end of the cannulated probe is in engagement with the distal end of the elongate member, wherein a distal portion of the cannulated probe carries a pair of bipolar electrodes and advancing the cannulated probe delivers the pair of bipolar electrodes to a treatment location proximate the basivertebral nerve.

In Example 28, a system for treating a basivertebral nerve in a vertebral body of a patient, the system comprising an introducer having a central channel, the introducer configured to pass through the cortical bone of the vertebral body and into the cancellous bone of the vertebral body, a steerable assembly defining a proximal end and a distal end, the steerable assembly having a handle disposed at the steerable assembly proximal end, the steerable assembly sized for passage through the central channel of the introducer, the steerable assembly comprising an elongate member having a distal end comprising a distal portion, a hypotube slidably received about and removable from the elongate member, the hypotube comprising a distal end engageable with the distal end of the elongate member, an actuator coupled with the handle and operable to impart an axial force to one of the elongate member and the hypotube relative to the other of the elongate member and the hypotube to cause the distal end of the steerable assembly to move from a first configuration to a deflected configuration to form a path within the cancellous bone of the vertebral body toward the basivertebral nerve.

In Example 29, the system of Example 28, wherein the shaft further comprises a first conductor coupled with the first electrode and configured for electrical communication with a first pole of a radiofrequency energy generator comprises a first electrode disposed at the distal end of the steerable assembly, a second electrode disposed at the distal end of the steerable assembly, and a second conductor coupled with the second electrode and configured for electrical communication with a second pole of the radiofrequency energy generator.

In Example 30, the system of Example 29, wherein the first electrode is disposed at the distal portion of the elongate member, and wherein the second electrode comprises a conductive portion of the hypotube.

In Example 31, the system of Example 29, wherein the first and second electrodes are carried by the hypotube.

In Example 32, the system of Example 29, wherein the distal portion of the elongate member comprises a bulb welded to the distal portion of the elongate member proximal from a sharpened distal tip.

In Example 33, the system of Example 28, wherein the actuator comprises a bail actuator configured to be toggled between an inactive configuration, in which no axial force is applied between the elongate member and the hypotube, and an active configuration, in which the axial force is applied to one of the elongate member and the hypotube relative to the other of the elongate member and the hypotube, and toggling of the actuator to the active configuration comprises proximally retracting the elongate member relative to the hypotube.

In Example 34, the system of Example 28, wherein the handle further comprises a removable portion coupled with a proximal end of the hypotube, the removable portion comprising a locking member configured to releasably couple the removable portion with the handle.

In Example 35, the system of Example 28, further comprising a cannulated probe sized for passage through the central channel of the introducer, the cannulated probe configured to be slidably received over the elongate member, the cannulated probe having a distal end engageable with the distal end of the elongate member wherein at least one electrode is located on the cannulated probe distal end.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a schematic illustration of vertebral levels and vertebrae that can be treated by systems and methods consistent with various aspects of the present disclosure.

FIG. 2 is a schematic illustration of a medical device with a steerable assembly for treating a vertebral body, consistent with various aspects of the present disclosure.

FIG. 3 is a cross-sectional view of a steerable tip in a rested configuration, consistent with various aspects of the present disclosure.

FIG. 4 is a side view of the steerable tip in a strained configuration, consistent with various aspects of the present disclosure.

FIG. 5A is a partially sectioned view of a steerable assembly in an unlocked and rested configuration, consistent with various aspects of the present disclosure.

FIG. 5B is a partially sectioned view of the steerable assembly in a locked and strained configuration, consistent with various aspects of the present disclosure.

FIG. 6 is cross-sectional view of an alternative steerable tip, consistent with various aspects of the present disclosure.

FIG. 7 is a side view of an alternative guidewire in a rested configuration, consistent with various aspects of the present disclosure.

FIG. 8 is a side view of an alternative guidewire in a rested configuration, consistent with various aspects of the present disclosure.

FIGS. 9A-9C are different views of a hypotube. More specifically, FIG. 9A is a side view of the hypotube in a rested configuration, consistent with various aspects of the present disclosure. FIG. 9B is a cross-sectional view of the hypotube as indicated by line 8B-8B in FIG. 9A, consistent with various aspects of the present disclosure.

FIG. 9C is a side view of the hypotube in a strained configuration, consistent with various aspects of the present disclosure.

FIG. 10 is a side view of an alternative guidewire in a rested configuration, consistent with various aspects of the present disclosure.

FIG. 11 is a side view of a probe, consistent with various aspects of the present disclosure.

FIG. 12 is a flowchart of a method of accessing and treating tissue within a vertebral body, consistent with various aspects of the present disclosure.

FIGS. 13A-13G are a series of top views of operations of accessing and treating tissue within a vertebral body, consistent with various aspects of the present disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

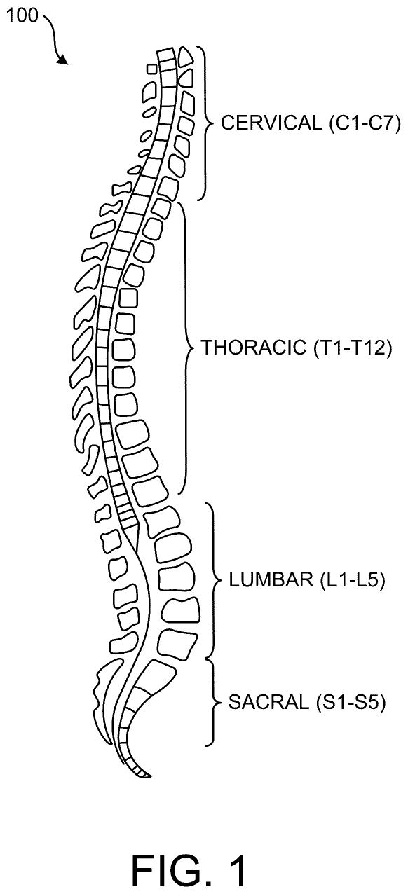

FIG. 1 shows a human spine 100 including vertebral levels and vertebral bodies that can be treated by systems and methods consistent with various aspects of the present disclosure. Treatment procedures may include modulation of nerves within or surrounding bones. The terms “modulation” or “neuromodulation”, as used herein, shall be given their ordinary meaning and shall also include ablation, permanent denervation, temporary denervation, disruption, blocking, inhibition, electroporation, therapeutic stimulation, diagnostic stimulation, inhibition, necrosis, desensitization, or other effect on tissue. Neuromodulation shall refer to modulation of a nerve (structurally and/or functionally) and/or neurotransmission. Modulation is not necessarily limited to nerves and may include effects on other tissue, such as tumors or other soft tissue.

Embodiments described herein are directed to systems and methods for modulating nerves within or adjacent (e.g., surrounding) bone. In some embodiments, an intraosseous nerve (e.g., basivertebral nerve (BVN)) within a vertebral body of the spine is modulated for treatment or prevention of chronic back pain. The vertebral body may be located in any level of the vertebral column (e.g., cervical, thoracic, lumbar and/or sacral). Multiple vertebral bodies may be treated in a single visit or procedure (simultaneously or sequentially). The multiple vertebral bodies may be located in a single spine segment or in different spine segments. Examples of the former are two adjacent vertebral bodies in the sacral spine segment (e.g., S1 and S2) or lumbar spine segment (e.g., L3, L4 and/or L5) or thoracic spine segment or cervical spine segment), and an example of the latter is an L5 vertebra in the lumbar spine segment and an S1 vertebra in the sacral spine segment. Intraosseous nerves within bones other than vertebral bodies may also be modulated. For example, nerves within a humerus, radius, femur, tibia, calcaneus, tarsal bones, hips, knees, and/or phalanges can be modulated.

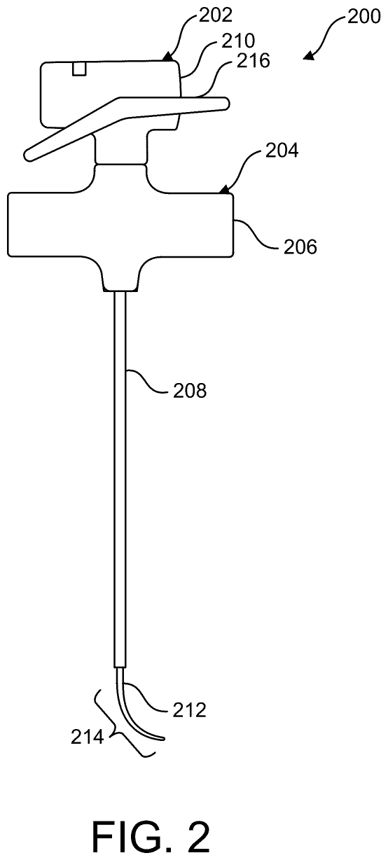

FIG. 2 shows a medical device 200 for treating a vertebral body. The medical device 200 is configured to access the vertebral body, position therapy components adjacent the treatment tissue (e.g., the basivertebral nerve (BVN)), and deliver sufficient radiofrequency energy to the nerve to modulate (e.g., ablate) the nerve so as to reduce or eliminate a patient's pain.

In the illustrated embodiment, the medical device 200 includes a steerable assembly 202 and an introducer assembly 204. The introducer assembly 204 includes a handle 206 at the proximate end and a straight introducer shaft 208 attached to the handle 206 and extending distally therefrom. The introducer assembly 204 includes a bore extending through the handle 206 and the shaft 208 that is configured to receive the steerable assembly 202.

In the illustrated embodiment, the steerable assembly 202 includes a steering actuator 210 at the proximate end, the distal side of which abuts the proximal side of the handle 206. In addition, the steering assembly 202 includes channeling components 212 that are attached to the steering actuator 210 and extend distally therefrom. The channeling components 212 extend through the bore in the introducer assembly 204 and emerge from the distal end of the shaft 208. The channeling components 212 have a curved configuration in the distal region 214. The channeling components 212 are also flexible in the distal region 214, such that the channeling components 212 can assume a straight configuration as they are traversing through the shaft 208 during the positioning of the steerable assembly 202 in the introducer assembly 204.

Furthermore, the steerable assembly 202 includes a lever 216 that can be actuated by a physician. The lever 216, when switched to an “active” or “strained” position, causes the channeling components 212 to curve more than the amount of pre-curvature that they naturally have at rest.

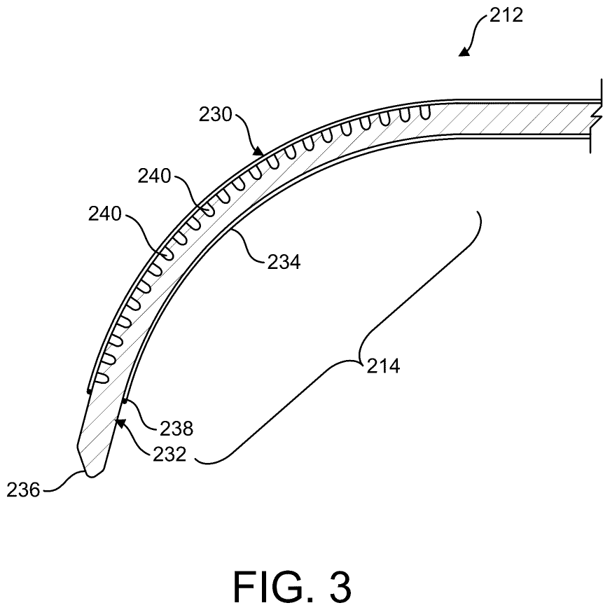

FIG. 3 shows a cross-sectional of a steerable guide assembly 230 in a rested or inactive configuration. The guide assembly 230 is the distal region 214 of the channeling components 212. In the illustrated embodiment, the guide assembly 230 includes a central guidewire 232 and a hypotube 234 surrounding the guidewire 232 along most of its length. The guidewire 232 has a distal end that emerges from the distal end of the hypotube 234 and has a sharpened chisel tip 236.

In certain embodiments, the hypotube 234 is welded to the guidewire 232 at a circumferential weld 238 at or near the distal end of the hypotube 234. As shown, proximal to the circumferential weld 238, the guidewire 232 includes an array of notches 240 that ends a distance proximal from the tip 236 that is, for example, near the proximal end of the curved portion of the guidewire 232 at rest. In some embodiments, the length of the array of notches 240 is ten to twenty-five diameters of the guidewire 232 long. In some embodiments, a length of the array of notches 240 is about 35 millimeters (mm) to about 50 mm long, and the outer diameter of the guidewire 232 is about 1.75 mm. In addition, the wall thickness of the hypotube 234 is about 0.25 mm, the inner diameter of the hypotube 234 is about 1.8-2.1 mm and the outer diameter is about 2.0-2.3 mm.

The notches 240 are transverse cuts that extend part of the way (e.g., halfway) through the guidewire 232 that make one side of the guidewire 232 (i.e., the top/left side, as shown in FIG. 3) axially weaker compared to the circumferentially opposite side (i.e., the bottom/right side, as shown in FIG. 3). As stated previously, the channeling components 212 can be selectively translated by the physician (shown in FIG. 2). Such translation generates a proximally-oriented axial force applied to the guidewire 232 by the steering actuator 210 (essentially pulling proximally on the weld 238) while an opposing, distally-oriented axial force is applied to the hypotube 232 also by the steering actuator 210 (essentially pushing distally on the weld 238). Such axial forces can be created by displacing the proximal end of the hypotube 234 about 1.2-2.1 mm with respect to the proximal end of the guidewire 232.

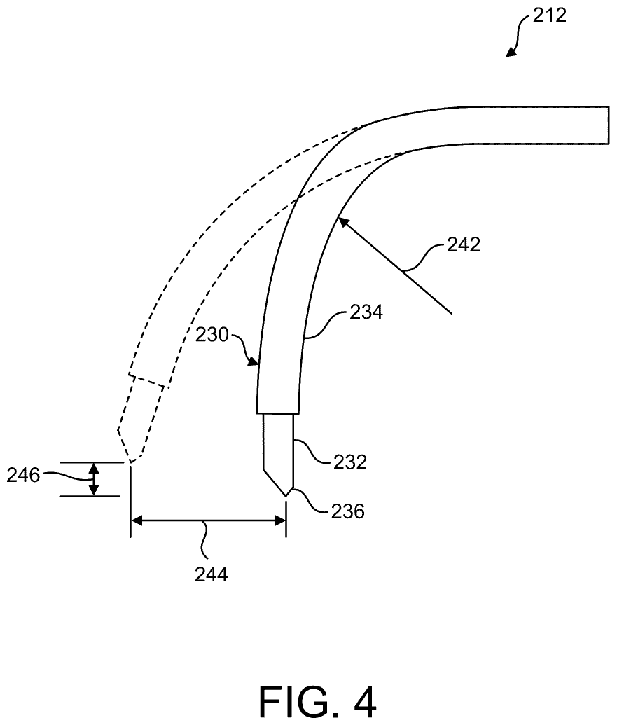

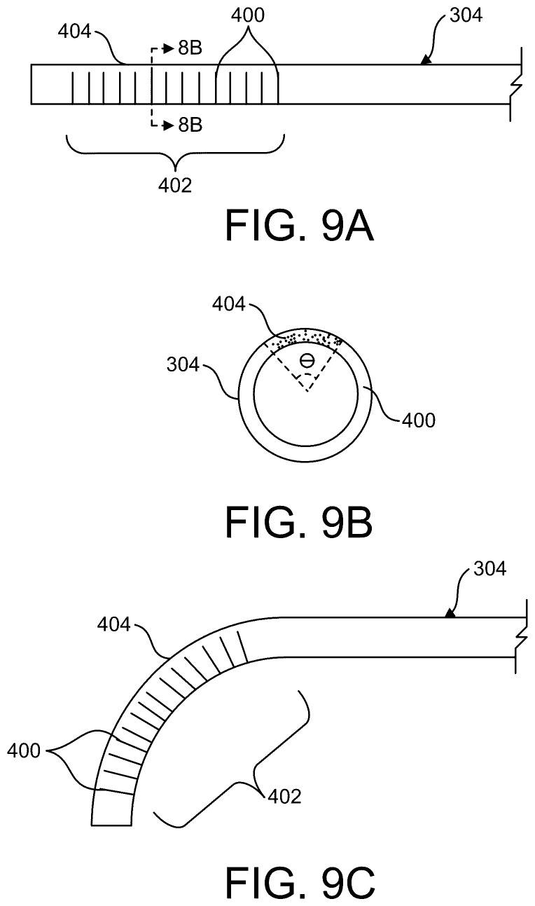

When an axial force is applied to the guidewire 232, the weaker side will not be able to resist the axial force as much as the stronger side, which causes the channeling components 212 to bend towards the stronger side. This effect is shown in FIG. 4, wherein the guide assembly 230 is in a deflected configuration. In some embodiments, the guide assembly 230 has a substantially decreased radius of curvature 242 compared to its rested configuration (shown in phantom in FIG. 4). This results in the tip 236 being displaced proximally by a substantial axial distance 244 and medially by a substantial transverse distance 246. In some embodiments, the radius of curvature of the rested configuration of the guide assembly 230 is about 31 mm, and the radius of curvature of the deflected configuration is about 14 mm. In some embodiments, the axial distance 244 is about 24 mm and the transverse distance is about 5.1 mm, and in other embodiments, the axial distance 244 is about 18 mm and the transverse distance is about 7.5 mm. These embodiments can result from the heel (shown in FIG. 9B) of the hypotube 234 being about seventy-five degrees with relative displacements between the proximal ends of the guidewire 232 and the hypotube 234 being 1.2 mm and 2.1 mm, respectively.

This deflection of the guide assembly 230 will assist in the channeling components 212 catching and making a tighter turn as they begin to exit the shaft 208 (shown in FIG. 2) and enter the vertebral body. The tighter turn can be the result of the lateral force applied to the to the vertebral body by the tip 236. In some embodiments, the lateral force from the guide assembly 230 in the rested configuration is about 7.5-10 Newtons (N). However, with relative displacement of 1.2 mm between the proximal ends of the guidewire 232 and the hypotube 234, the lateral force is about 22-24 N, and with relative displacement of 2.1 mm between the proximal ends of the guidewire 232 and the hypotube 234, the lateral force is about 32-34 N. These embodiments can result from the heel of the hypotube 234 being about seventy-five degrees.

Having a tighter turn into the vertebral body can be advantageous, for example, where the angle of approach (through the pedicle) is low and/or because the shaft 208 does not need to be inserted into the vertebral body as far. In addition, in some embodiments, the steering actuator 210 is configured to have multiple deflected configurations that can vary the radius of curvature 242 and the amounts of displacement distance 244, 246. In such embodiments, the tip 236 can be configured in positioned in one or more intermediate locations between the two that are shown in FIGS. 3 and 4. This gives the physician even more flexibility with regard to the placement of the shaft 208 with respect to the vertebral body. In addition, while the effects of deflection are shown with respect to guide assembly 230, the same or similar effects can occur with other embodiments of the present disclosure that follow.

In other embodiments, the distal end of the guidewire has a shoulder that is wider than the inner diameter of the hypotube (e.g., positioned at the location where the weld 238 is). In other words, the distal end of the guidewire that is beyond the distal end of the hypotube (i.e., the chisel tip) has a larger diameter than the proximal portion of the guidewire that is positioned in the hypotube. Thus, the hypotube can push on the shoulder on the proximal side of the tip when an axial force is applied by the steering actuator. In such embodiments, the guidewire is not welded to the hypotube and the force is created by pulling the shoulder against the distal end of the hypotube, which increases the curvature of the channeling components. As the guidewire and the hypotube are not permanently affixed to each other, the hypotube can be separated from the guidewire, for example, to replace the hypotube with another component (such as, for example, a therapy component).

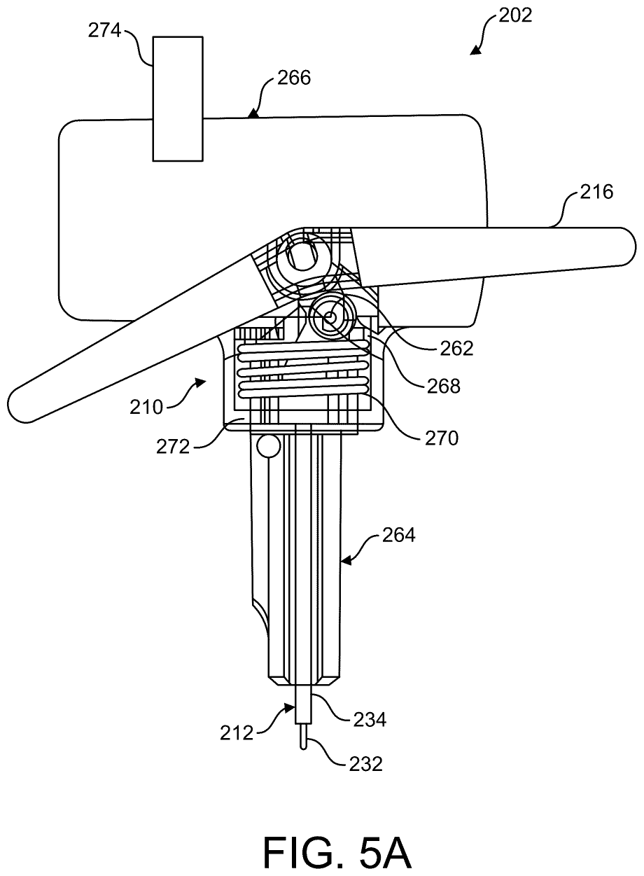

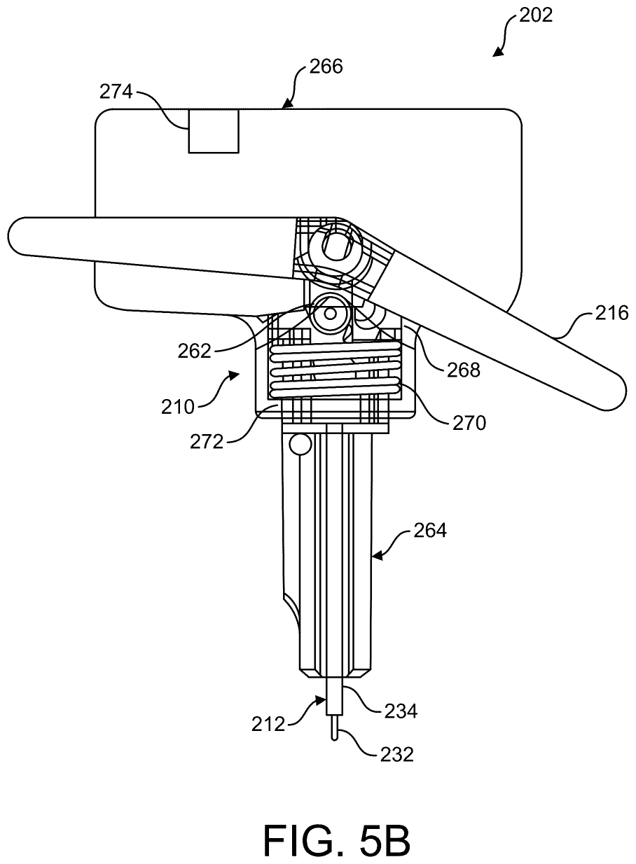

FIG. 5A shows the steerable assembly 202 in an unlocked and rested configuration, with selected sections showing the internals of the steerable assembly 202. As stated previously, the steerable assembly 202 includes the steering actuator 210 and the channeling components 212 (i.e., the guidewire 232 and the hypotube 234). In the illustrated embodiment, the steering actuator 210 includes a lever 216, a toggle linkage 262, a body 264, and a head 266. The lever 216 is rotatably connected to the head 266, and the switch is connected to the proximal end of the toggle linkage 262. The distal end of the toggle linkage 262 is rotatably connected to the body 264, and the body 264 is slidably connected to the head 266. The body 264 includes a shoulder 268 at the proximal end which is contacted by a spring 270 that also contacts a lip 272 in the head 266. Thereby, the spring 270 biases the body 264 proximally into the head 266, however, this biasing force is opposed by the toggle linkage 262.

In the rested configuration (as shown in FIG. 5A), the toggle linkage 262 is axially shorter than in the locked configuration (as shown in FIG. 5B). Thereby, when the switch is in the rested position, the body 264 is farther inside of the head 266 than when the lever 216 is in the deflected position (as shown in FIG. 5B).

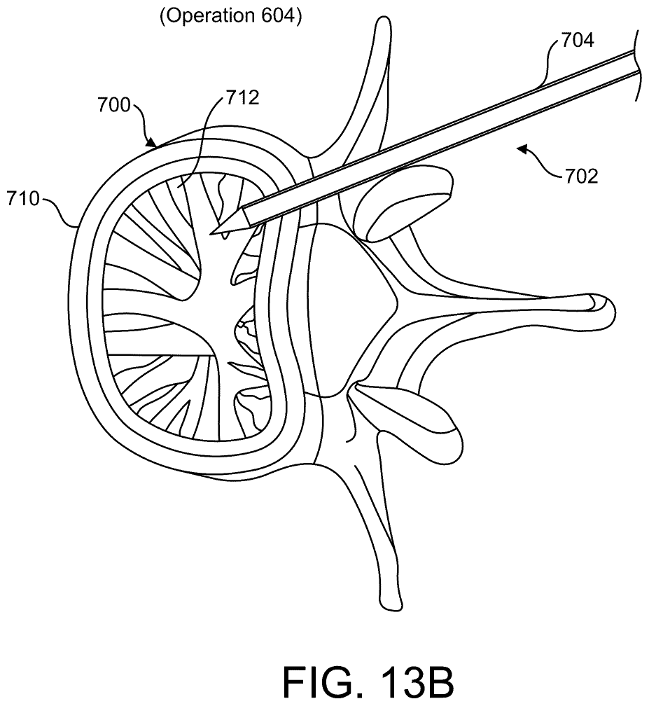

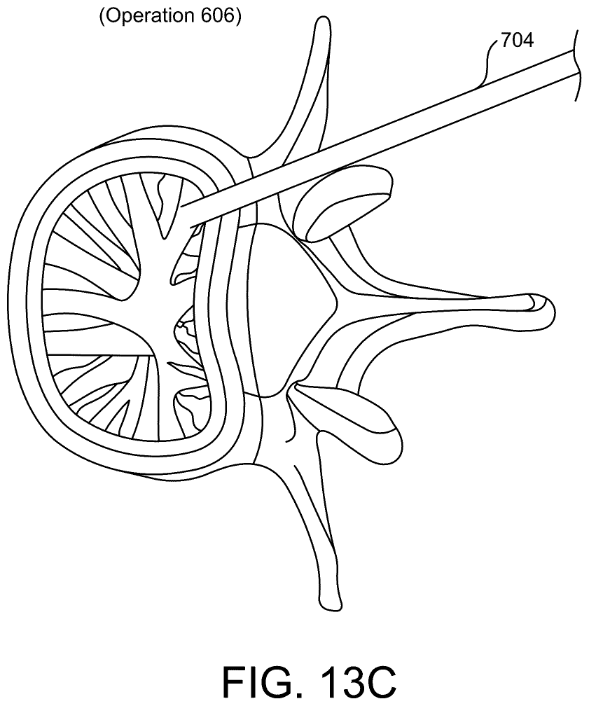

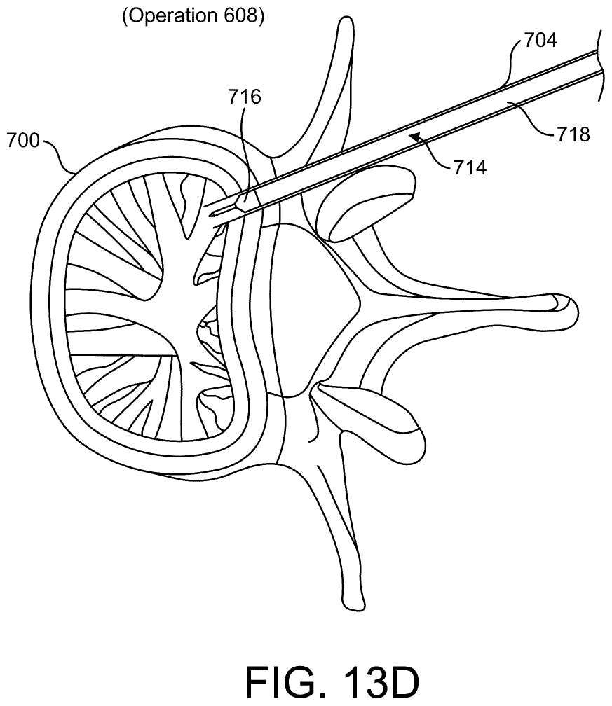

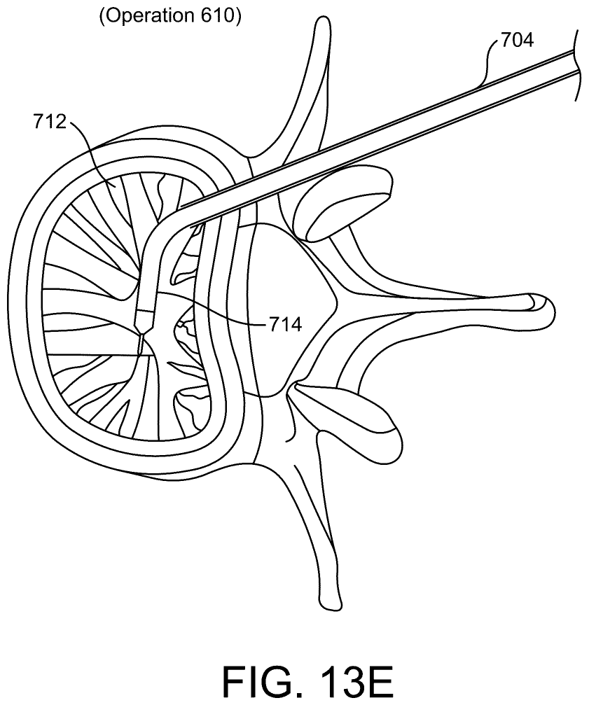

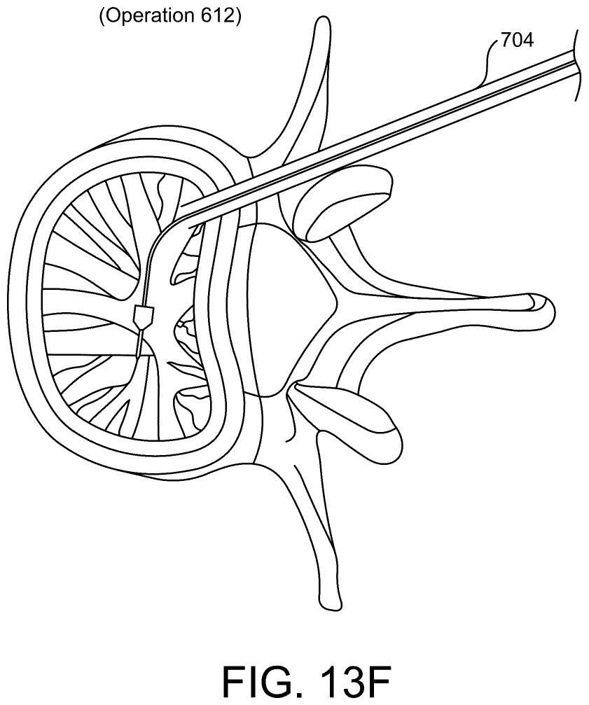

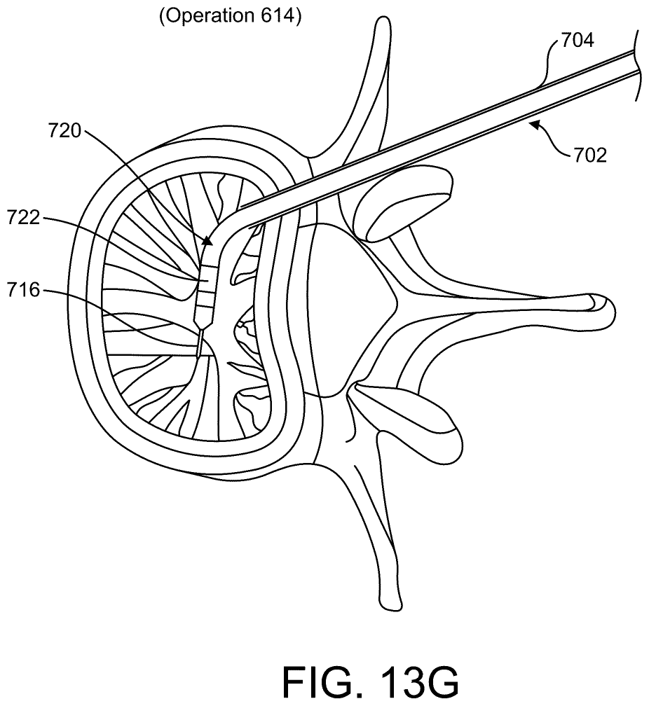

In the illustrated embodiment, the head 266 includes a locking tab 274, which is shown in the up or unlocked position in FIG. 5A. The locking tab 274 can be operatively connected to a securing feature (not shown) inside of the head 266 (e.g., a clamp) that selectively engages the guidewire 232. When the steerable assembly 202 is in the unlocked position, the steering actuator 210 is not connected to the guidewire 232. Thus, the steering actuator 210 cannot apply an axial force to the channeling components 212 when in the unlocked configuration. However, the steering actuator 210 can be removed from the guidewire 232 when in the unlocked configuration, which can be advantageous as explained with respect to FIG. 13F.

In the illustrated embodiment of FIG. 5B, the hypotube 234 is attached to the body 264, and the guidewire 232 has been locked to the head 266 by the physician moving the locking tab 274 into the down or locked position. When the lever 216 is moved to the deflected position, the body 264 moves distally with respect to the head 266. Thus, the proximal end of the hypotube 234 moves distally with respect to the proximal end of the guidewire 232. However, the distal end of the hypotube 234 is constrained from moving with respect to the distal end of the guidewire 232 by the weld 238, so the hypotube 234 is compressed and the guidewire 232 is tensioned, which causes the guide assembly 230 (shown in FIG. 4) to curve.

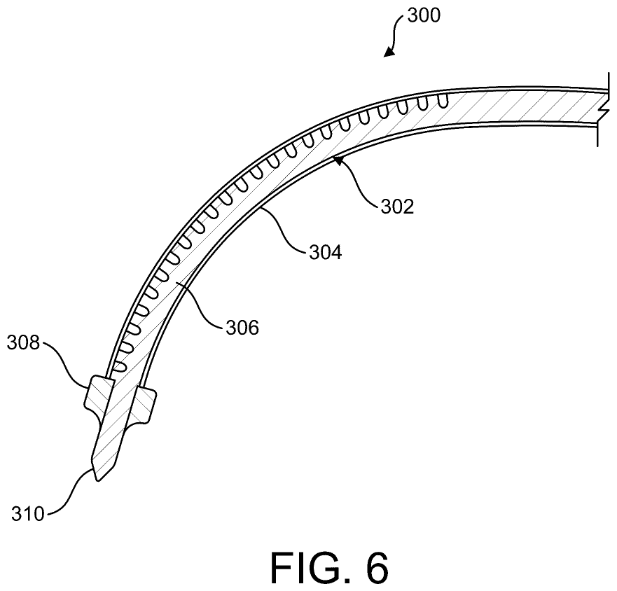

FIG. 6 shows an alternative steerable guide assembly 300. In the illustrated embodiment, guide assembly 300 includes a guidewire 302 and a hypotube 304 surrounding most of the guidewire 302. The guidewire 302 includes a wire 306 positioned mostly inside of the hypotube 304 and a bulb 308 connected (e.g., welded) to a thinned portion of the wire 306. The bulb 308 is located proximal to the sharpened end 310 of the wire 306 (e.g., between one and five diameters of the bulb 308). The arrangement of the wire 306 extending from the bulb 308 forms a narwhal tip (i.e., a tip with a smaller diameter segment adjacent to a larger diameter segment that is proximal therefrom). The guidewire 302 slidably receives hypotube 304, but the outer diameter of the bulb 308 is larger than the inner diameter of the hypotube 304. Thereby, the hypotube 304 cannot pass over the narwhal tip when an axial force is applied to the hypotube 304 by the guidewire 302.

Furthermore, the outer diameter of the bulb 308 is larger than the outer diameter than the hypotube 304. Thereby, the bulb 308 can clear a path through vertebral body for the hypotube 304. In addition, the decreased size of the end 310 allows the guidewire 302 to bite into the vertebral body more easily than if the end had a diameter similar to that of the hypotube 304 or bulb 308. Thereby, the guide assembly 300 can have a tighter curvature in the vertebral body. Another advantage of the narwhal tip is that the bulb 308 prevents the end 310 from contacting the interior of the shaft 304 (shown in FIG. 2), which prevents skiving or other damage when the steerable assembly 202 is inserted into the introducer assembly 204.

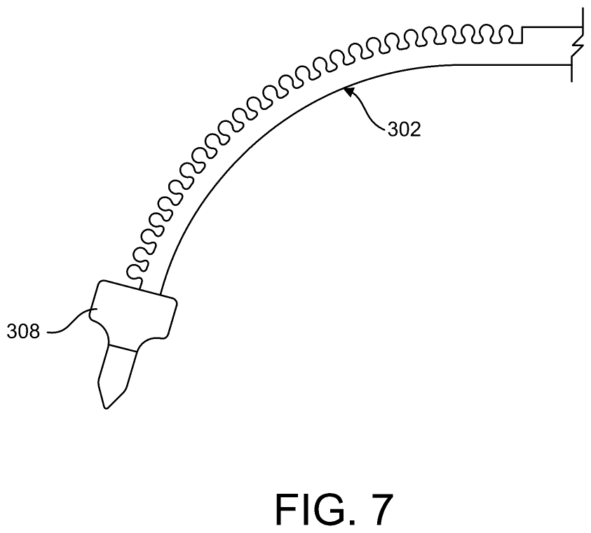

FIG. 7 shows an alternative guidewire 302 in a rested configuration. In the illustrated embodiment, the guidewire 302 is naturally pre-curved (although in other embodiments, the guidewire 302 can be straight). In some embodiments, the guidewire 302 can have a similar configuration and dimensions to that of guidewire 232 (shown in FIG. 3), although the bulb 308 is welded to the guidewire 302 instead of having the hypotube 304 (shown in FIG. 6) welded to the guidewire 302. When a relative axial force is applied to the guidewire 302 and the hypotube 304, the distal end of the hypotube 304 pushes against the proximal side of the bulb 308. Thus, the increased curvature from the rested configuration can be achieved without permanently affixing the guidewire 302 to the hypotube 304, so the hypotube 304 can be removed from off of the guidewire 302 while the guidewire 302 is positioned in a vertebral body.

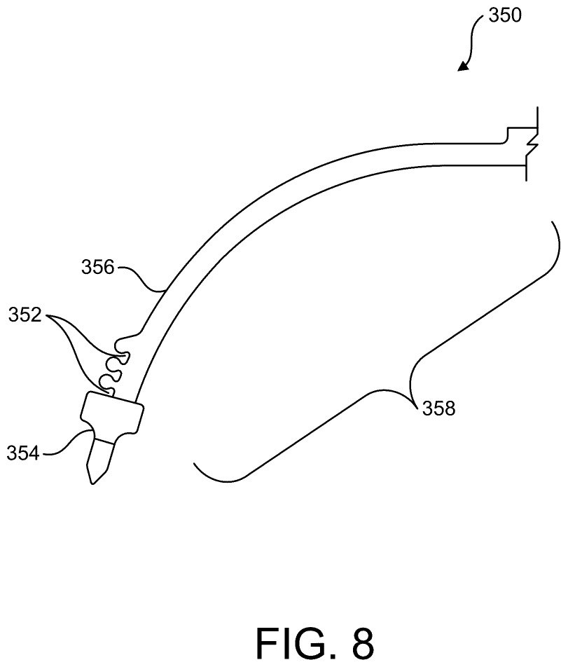

FIG. 8 shows an alternative guidewire 350 in a rested configuration. In the illustrated embodiment, the guidewire 350 has a low number of small notches 352 (e.g., three) near a narwhal tip 354. However, there is also a large notch 356 proximal from the small notches 352 that occupies at least three-quarters of the length of a distal region 358 of the guidewire 350, in some embodiments. Since most of the material exists on one side of the guidewire 350 in the distal region 358 (i.e., the bottom side, as shown in FIG. 8), the distal region 358 will curve when an axial force is applied (shown in FIG. 6).

FIG. 9A is a side view of hypotube 304 in a rested configuration where the hypotube 304 is naturally straight (although in other embodiments, the hypotube 304 can be pre-curved). In the illustrated embodiment, a substantial portion (or all) of hypotube 304 is made from metal (e.g., stainless steel), and the hypotube 304 includes an array of slits 400 in a distal region 402. In some embodiments, the array of slits 400 begins about 0.50 mm proximal of the distal end of the hypotube 304 and extends for about 38 mm up the hypotube 304. Slits 400 allow one side of the hypotube 304 (i.e., the bottom side, as shown in FIGS. 9A-9C) to be shortened compared to the circumferentially opposite side (which is solid and is known as a heel 404) without plastically deforming the hypotube 304. Thus, the hypotube 304 can curve in the same way as the guidewire 302 (shown in FIG. 6.)

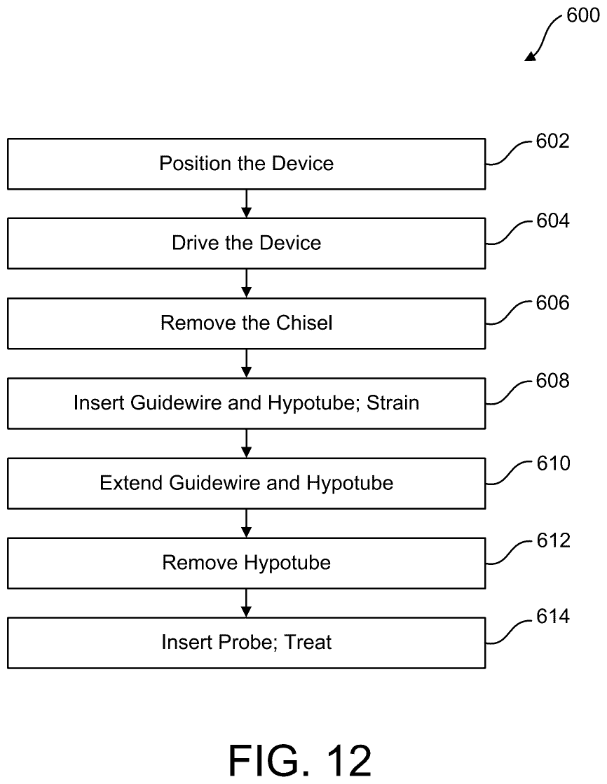

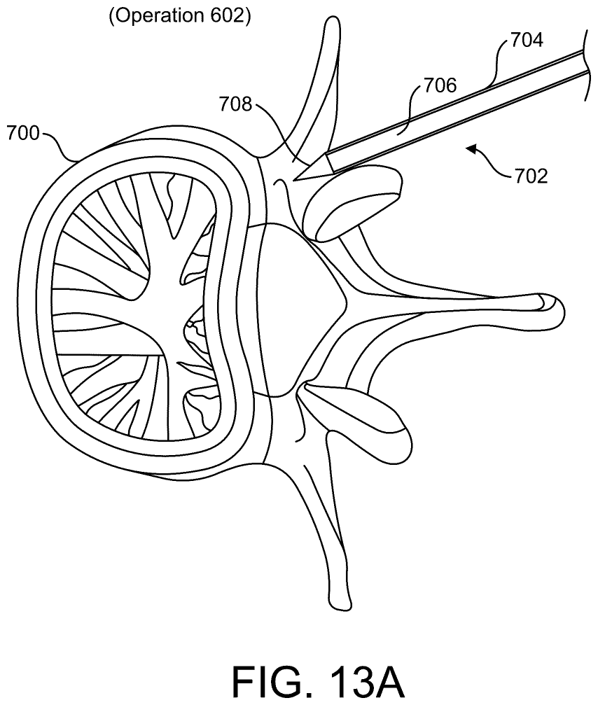

FIG. 9B is a cross-sectional view of the hypotube 304 as indicated by line 8B-8B in FIG. 9A (i.e., through one of the slits 400). In the illustrated embodiment, the slits 400 are made through the majority of circumference of the hypotube 304. The slits 400 can be made using a laser, for example, and can have ends that are oriented radially with respect to the central axis of the hypotube 304. In some embodiments, the heel 404 (i.e., the remaining portion of material) subtends about a seventy-five degree angle θ or less; in some embodiments, the heel 404 subtends about a forty-five degree angle θ or more; and in some embodiments, the heel 404 subtends about a sixty degree angle θ. The heel 404 is sized large enough to handle the compressive axial force put on the hypotube 304 during insertion, for example, at operations 608 and 610 (shown in FIG. 12), and any radial or twisting forces that may occur during use, for example, when routing the medical device 302 through a patient at operation 202. In some embodiments, the slits 400 are made in an interlocking pattern.

FIG. 9C is a side view of the hypotube 304 in a deflected configuration. The slits 400 are narrow in axial length but are wide enough and numerous enough to permit the appropriate amount of curvature to match that of the guidewire 302. The size and number of the slits 400 can influence how tightly the hypotube 304 curves when an axial force is applied (i.e., the larger and more numerous the slits 400 are, the tighter the curvature).

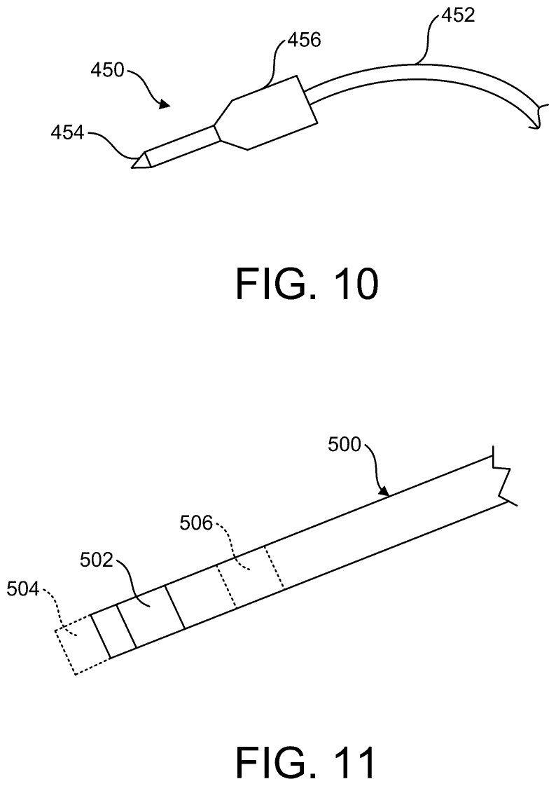

FIG. 10 shows an alternative guidewire 450 in a rested configuration. In the illustrated embodiment, the guidewire 450 is pre-curved, so in a rested (or inactive) configuration, it is naturally bent. In some embodiments, about 41 mm of the distal end of the guidewire 450 is curved before straightening out. The guidewire 450 includes a central wire 452 with a sharpened end 454, and guidewire 450 also includes a bulb 456 connected (e.g., welded) to the wire 452. The bulb 456 is located proximally up from the end 454 (e.g., between one and five diameters of the bulb 456), so the guidewire 450 has a narwhal tip (i.e., a distal end with a smaller diameter segment distally adjacent to a larger diameter segment that is proximal therefrom). In some embodiments, the diameter of the wire 452 is about 1.3-1.4 mm, the outer diameter of the bulb 456 is about 2.1-2.4 mm, the length of the bulb 456 is about 3.4 mm, and the bulb 456 is positioned about 3.8 mm from the distal end of the wire 452. While the guidewire 450 does not have the imbalance of material on one side of the wire 452 that other embodiments have (e.g., wire 306), when the hypotube 304 (shown in FIG. 6) is forced against the guidewire 450, the guidewire 450 will bend due to the slits 400, which will collapse under tension.

FIG. 11 is a side view of a probe 500. In the illustrated embodiment, the probe 500 is a therapy component that replaces the hypotube 304 (shown in FIG. 9A) after the guidewire 450 (shown in FIG. 10) has been positioned in the vertebral member. The probe 500 is an elongate hollow member that includes a ring electrode 502. The probe 500 also includes electrical insulation (not shown) lining the interior of the probe 500 to electrically separate the electrode 502 from the guidewire 450 and from other electrical components, such as, for example, a thermocouple. The electrical insulation exists so that the guidewire 450 can work with the probe 500 in a bipolar manner (i.e., the guidewire 450 servers as the second electrode). Once the distal tip of the guidewire 450 and the electrode 502 are properly positioned in the vertebral member, for example, 1-2 mm anterior to the BVN, treatment can commence by passing radiofrequency electrical energy between the guidewire 450 and the electrode 502 to ablate the BVN.

In the illustrated embodiment, the probe 500 is naturally straight at rest and bends as it is inserted along the guidewire 450, although in other embodiments, the probe 500 is also pre-curved. However, in such embodiments, the probe 500 would be straight when positioned inside of the introducer 304. In some embodiments, the probe 500 can have a distal region with slits similar to the slits 400 (shown in FIG. 9A), and/or the probe 500 can be made of more flexible material than the hypotube 304 is.

In the illustrated embodiment, the electrode 502 is spaced proximally up from the distal end of the probe 500 so as to avoid making an electrical connection with the bulb 456 (shown in FIG. 10). In some embodiments, the electrode 502 is positioned at the distal end of the probe 500, so an electrical insulator 504 (shown in phantom) is positioned between the probe 500 and the bulb 456. In some embodiments, the electrical insulator 504 is be connected to the probe 500, but in other embodiments, the electrical insulator 504 is connected to the guidewire 450. The electrical insulator could be formed of any biocompatible insulating material including, for example, a polyether block amide. Furthermore, in some embodiments, the probe 500 includes a second ring electrode 506 (shown in phantom) that is spaced apart and electrically isolated from electrode 502. Such embodiments are bipolar due to electrodes 502, 506, and they do not use the guidewire 450 as an electrode. In yet other embodiments, there is only a single electrode 502 and the guidewire 450 is not used as an electrode. Such embodiments would be monopolar systems.

In some embodiments, the probe 500 has similar or the same dimensions to that of the hypotube 304. Thus, the outer diameter of the bulb 456 is larger than the outer diameter of the probe 500. Thereby, the bulb 456 can clear a path through vertebral body for the probe 500. This can be advantageous, for example, since the probe 500 can have sensitive components (e.g., the electrode 502 and thermocouples) that could be damaged if the probe 500 was driven through intact portions of the vertebral body.

FIG. 12 shows a flowchart of the method 600 of accessing and treating tissue within a vertebral body 700 using a medical device 702. FIGS. 13A-13G show a series of operations of accessing and treating tissue within the vertebral body 700 using the medical device 702. FIGS. 12 and 13A-13G will now be discussed in conjunction with one another, and each operation of the method 600 is illustrated by a corresponding one of FIGS. 13A-13G.

In the illustrated embodiment, the method 600 begins at the operation 602, wherein the medical device 702 is positioned against the vertebral body 700. The medical device 702 can include any suitable combination of the embodiments of components described in the present disclosure, such as, for example, medical device 200. The medical device 702 includes an introducer 704 that is an open-ended hollow shaft. Inside the central channel of the shaft is a chisel 706 with a sharpened tip 708.

At the operation 604, a portion of the medical device 702 is driven into the vertebral body 700 (e.g., by malleting the medical device 702), through the cortical bone region 710, and into the cancellous bone region 712 far enough that the distal end of the introducer 704 is located in the cancellous bone region 712.

At the operation 606, the chisel 706 is removed from the introducer 704.

At the operation 608, a steerable guide assembly 714 is inserted into the introducer 704. The guide assembly 714 includes a guidewire 716 and a hypotube 718 that surrounds most of the guidewire 716. Once the guide assembly 714 reaches the distal end of the introducer 704, the guide assembly 714 is forced to curve medially with respect to the vertebral body 700.

At the operation 610, the guide assembly 714 is extended from the introducer 704 into the cancellous bone region 712 (e.g., by malleting the medical device 702). As the guide assembly 714 is extended from the introducer it guides or channels through the cancellous bone toward the treatment region.

At the operation 612, the hypotube 718 is removed from the introducer 304 while the guidewire 716 remains in place.

At operation 614, a probe 720 is passed inside of the introducer 704 and over the guidewire 716. In the illustrated embodiment, the probe 720 includes an electrode 722 and electrical insulation, for example, lining the interior of the probe 720 to electrically separate the electrode 722 from the guidewire 716 and from other electrical components, such as, for example, a thermocouple. This is because the medical device 702 is bipolar, and the guidewire 716 servers as the other electrode. Once the distal tip of the guidewire 716 and the electrode 722 are properly positioned, for example, 1-2 mm anterior to the BVN, treatment can commence by passing radiofrequency electrical energy between the guidewire 716 and the electrode 722 to ablate the BVN.

In some alternate embodiments, operations 608 and 610 are performed with the probe 720 over the guidewire 716 instead of the hypotube 718. In such embodiments, the operation 612 can be absent and the method 600 can advance directly from operation 610 to the treatment portion of operation 614. In some alternate embodiments, the electrode 502 is positioned at the distal end of the probe 500 and/or the distal end of the probe 500 is not otherwise electrically insulated from the electrode 502, so the method 600 can be modified. For example, at operation 614, the probe 500 can be inserted until contacting the proximal end of the narwhal tip and then proximally retracted by a small distance. This creates an electrically insulating air gap between the electrode 502 and the narwhal tip, so an extra electrically insulating components are not required.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A system for treating a basivertebral nerve in a vertebral body of a patient, the system comprising:
an introducer having a central channel, the introducer configured to pass through the cortical bone of the vertebral body and into the cancellous bone of the vertebral body;
a steerable assembly sized for passage through the central channel of the introducer, the steerable assembly having a first configuration and a deflected configuration, the steerable assembly including:
an elongate member having a proximal end and a distal portion, wherein the distal portion of the elongate member is deflectable between the first configuration and the deflected configuration; and
a hypotube disposed over a portion of the elongate member, the hypotube comprising a proximal end and a distal end;
wherein the distal portion of the elongate member is configured to engage the distal end of the hypotube;
wherein axial translation of one of the elongate member and the hypotube relative to the other of the elongate member and the hypotube causes the steerable assembly to move from the first configuration to the deflected configuration.

2. The system of claim 1, wherein the hypotube is formed at least in part from stainless steel.

3. The system of claim 1, wherein:
the elongate member defines a plurality of notches at the elongate member distal portion to facilitate deflection of the distal portion of the elongate member between the first configuration and the deflected configuration; and
the distal portion of the elongate member defines a longitudinal axis and first and second cylindrical sections defined by a plane extending along the longitudinal axis, and wherein the plurality of notches are defined in the first cylindrical section.

4. The system of claim 1, wherein the distal portion of the elongate member defines a shoulder and wherein the distal end of the hypotube engages the shoulder of the elongate member.

5. The system of claim 1, wherein the distal end of the elongate member has a pre-formed curve in the first configuration.

6. The system of claim 1, further comprising a first electrode and a second electrode configured to deliver bipolar radiofrequency energy to a treatment location.

7. The system of claim 6, further comprising:
a cannulated probe sized for passage through the central channel of the introducer, the cannulated probe configured to be slidably received over the elongate member, the cannulated probe having a proximal end and a distal end;
wherein the first electrode and the second electrode are located proximate the cannulated probe distal end.

8. The system of claim 6, wherein the elongate member comprises the first electrode and wherein the hypotube comprises the second electrode.

9. A method of treating a basivertebral nerve in a vertebral body of a patient, the method comprising:
inserting an introducer through the cortical bone of the vertebral body and into a cancellous bone region of the vertebral body, the introducer having a shaft defining a central channel and an axial opening in communication with the central channel located at a distal end of the shaft;
advancing a steerable assembly through the central channel of the introducer, the steerable assembly comprising:
an elongate member having a proximal end and a deflectable distal portion; and
a hypotube that extends over the elongate member such that a distal end of the hypotube is in engagement with the distal portion of the elongate member;
extending the steerable assembly beyond the axial opening of the introducer shaft; and
axially moving one of the elongate member and the hypotube relative to the other of the elongate member and the hypotube to cause the distal deflectable sections of the elongate member and the hypotube to deflect away from a central axis of the introducer shaft.

10. The method of claim 9, wherein the operation of axially moving one of the elongate member and the hypotube relative to the other of the elongate member and the hypotube causes the distal deflectable section of the elongate member to channel within the cancellous bone region.

11. The method of claim 9, wherein the operation of axially moving one of the elongate member and the hypotube relative to the other of the elongate member and the hypotube comprises pulling the elongate member in a proximal direction relative to the hypotube.

12. The method of claim 9, further comprising:
retracting the hypotube through the central channel of the introducer over the elongate member; and
advancing a cannulated probe through the central channel of the introducer, over the elongate member, and along a path in the cancellous bone region until a distal end of the cannulated probe is in engagement with the distal end of the elongate member, wherein a distal portion of the cannulated probe carries a pair of bipolar electrodes and advancing the cannulated probe delivers the pair of bipolar electrodes to a treatment location proximate the basivertebral nerve.

13. A system for treating a basivertebral nerve in a vertebral body of a patient, the system comprising:
an introducer having a central channel, the introducer configured to pass through the cortical bone of the vertebral body and into the cancellous bone of the vertebral body;
a steerable assembly defining a proximal end and a distal end, the steerable assembly having a handle disposed at the steerable assembly proximal end, the steerable assembly sized for passage through the central channel of the introducer, the steerable assembly comprising:
an elongate member having a distal portion comprising a distal tip;
a hypotube slidably received about and removable from the elongate member, the hypotube comprising a distal end engageable with the distal end of the elongate member;
an actuator coupled with the handle and operable to impart an axial force to one of the elongate member and the hypotube relative to the other of the elongate member and the hypotube to cause the distal end of the steerable assembly to move from a first configuration to a deflected configuration to form a path within the cancellous bone of the vertebral body toward the basivertebral nerve.

14. The system of claim 13, wherein the shaft further comprises:
a first electrode disposed at the distal end of the steerable assembly;
a first conductor coupled with the first electrode and configured for electrical communication with a first pole of a radiofrequency energy generator;
a second electrode disposed at the distal end of the steerable assembly; and
a second conductor coupled with the second electrode and configured for electrical communication with a second pole of the radiofrequency energy generator.

15. The system of claim 14, wherein the first electrode is disposed at the distal portion of the elongate member, and wherein the second electrode comprises a conductive portion of the hypotube.

16. The system of claim 14, wherein the first and second electrodes are carried by the hypotube.

17. The system of claim 14, wherein the distal tip of the elongate member comprises a bulb welded to the distal portion of the elongate member proximal from a sharpened distal tip.

18. The system of claim 14, wherein:
the actuator comprises a bail actuator configured to be toggled between an inactive configuration, in which no axial force is applied between the elongate member and the hypotube, and an active configuration, in which the axial force is applied to one of the elongate member and the hypotube relative to the other of the elongate member and the hypotube; and
toggling of the actuator to the active configuration comprises proximally retracting the elongate member relative to the hypotube.

19. The system of claim 13, wherein the handle further comprises a removable portion coupled with a proximal end of the hypotube, the removable portion comprising a locking member configured to releasably couple the removable portion with the handle.

20. The system of claim 13, further comprising:
a cannulated probe sized for passage through the central channel of the introducer, the cannulated probe configured to be slidably received over the elongate member, the cannulated probe having a distal end engageable with the distal end of the elongate member;
wherein at least one electrode is located on the cannulated probe distal end.
